(19) United States
(12) Patent Application Publication
(10) Pub. No.: US 2024/0263138 A1
(43) Pub. Date: Aug. 8, 2024

(54) METHODS FOR PREPARING A FOOD INGREDIENT AND COMPOSITIONS PRODUCED THEREBY

(71) Applicant: SuperMeat the Essence of Meat Ltd., Ness Ziona (IL)

(72) Inventors: Ido SAVIR, Herzliya (IL); Yuval LEVY PERETZ, Rehovot (IL); Tomer HALEVY, Nahariya (IL); Rivi HALIMI, Yavne (IL)

(73) Assignee: SuperMeat the Essence of Meat Ltd., Ness Ziona (IL)

(21) Appl. No.: 18/638,725

(22) Filed: Apr. 18, 2024

Related U.S. Application Data
(63) Continuation of application No. PCT/IL2022/051101, filed on Oct. 18, 2022.
(60) Provisional application No. 63/256,684, filed on Oct. 18, 2021.

(51) Int. Cl. C12N 5/077; A23L 13/00

(52) U.S. Cl. CPC C12N 5/0653; A23L 13/00; C12N 2500/30; C12N 2506/02

(57) ABSTRACT

A method of producing a food ingredient is provided. The method comprising: (a) providing stem cells; and (b) culturing the stem cells in the presence of an affective amount of fatty acids or precursor thereof selected to reach an intracellular fatty acid profile of an edible species of interest which is not of the stem cells, so at to result in a food ingredient having a lipid organoleptic property of the edible species.

### RELATED APPLICATIONS

This application is a Continuation of PCT Patent Application No. PCT/IL2022/051101 having International filing date of Oct. 18, 2022, which claims the benefit of priority under 35 USC § 119(e) of U.S. Provisional Patent Application No. 63/256,684 filed on Oct. 18, 2021. The contents of the above applications are all incorporated by reference as if fully set forth herein in their entirety.

### FIELD AND BACKGROUND OF THE INVENTION

The present invention, in some embodiments thereof, relates to methods for preparing a food ingredient and compositions produced thereby.

Cultured meat, also called synthetic meat, cell-cultured meat, clean meat, and in vitro meat, is meat grown in cell culture instead of inside animal, see Verbeke, Wim, Pierre Sans, and Ellen J. Van Loo. “Challenges and prospects for consumer acceptance of cultured meat.” Journal of Integrative Agriculture 14.2 (2015): 285-294, which is incorporated herein as a reference. Several patent documents describe a variety of cultured meat consumable products and related method: US patent app. Nos. 2005010965, 2006121006, and 2006029922 disclose process for production and cultured meat which comprises muscle cells that are grown ex vivo attached to either 2D or 3D support structure and further comprises other cells such as fat cells or cartilage cells, or both, that are grown ex vivo together with the muscle cells. The visual appearance (including color), smell, texture taste and price-per-product of the cultured meat published in the art is not yet satisfying, see Post, Mark J. “Cultured meat from stem cells: Challenges and prospects.” Meat Science 92.3 (2012): 297-301; Verbeke, Wim, et al. “′Would you eat cultured meat?′: Consumers′reactions and attitude formation in Belgium, Portugal and the United Kingdom.” Meat science 102 (2015): 49-58; and Forgacs, Gabor, Francoise Marga, and Karoly Robert Jakab. “Engineered comestible meat.” U.S. Pat. No. 8,703,216. 22 Apr. 2014, all incorporated herein as a reference.

Fat is the most important sensory component of edible meat. The influence of palatability on appetite and food intake in humans has been investigated in several studies: taste and smell are the main drivers to determine food acceptance, followed by vision, touch and audition. Sensory properties and perceived fattiness are criterias often used to determine contentment with a variety of produce. Another element of the quest for the perfect alternative meat is an ingredient's ability to melt at different temperatures or densities.

It therefore remains a long felt and unmet need to provide a novel fat cells production method which is more cost-efficient than the current methods of production and non-meat products which have improved meaty flavor.

### RELATED BACKGROUND ART

Lisitsyn et al. Foods and Raw Materials 2017, vol. 5, no. 2, pp. 54-61

### SUMMARY OF THE INVENTION

According to an aspect of some embodiments of the present invention there is provided a method of producing a food ingredient, the method comprising:
(a) providing stem cells; and
(b) culturing the stem cells in the presence of an affective amount of fatty acids or precursor thereof selected to reach an intracellular fatty acid profile of an edible species of interest which is not of the stem cells, so at to result in a food ingredient having a lipid organoleptic property of the edible species.

According to some embodiments of the invention, the stem cells are pluripotent stem cells.

According to some embodiments of the invention, the stem cells are embryonic stem cells.

According to some embodiments of the invention, the stem cells are induced pluripotent stem (iPS) cells.

According to some embodiments of the invention, the stem cells are adult stem cells.

According to some embodiments of the invention, the adult stem cells are multipotent stem cells.

According to some embodiments of the invention, the adult stem cells are of the mesoderm lineage.

According to some embodiments of the invention, the adult stem cells are mesenchymal stem cells.

According to some embodiments of the invention, the stem cells are selected from the group of avian stem cells, bovine stem cells, porcine stem cells, goat stem cells, sheep stem cells, shrimp stem cells and fish stem cells.

According to some embodiments of the invention, the avian stem cells are selected from the group of chicken stem cells and duck stem cells.

According to some embodiments of the invention, the stem cells are in suspension.

According to some embodiments of the invention, the stem cells are single cells.

According to some embodiments of the invention, the stem cells are cell aggregates or microtissues.

According to some embodiments of the invention, the culturing comprises a priming step followed by a differentiation step.

According to some embodiments of the invention, a concentration of fatty acids in a medium of the priming step is low to ensure commitment to a mesodermal lineage or adipogenic fate but still maintains the stem cells in a proliferative stage.

According to some embodiments of the invention, the concentration does not exceed 100 PM.

According to some embodiments of the invention, the priming step is 1-4 weeks long.

According to some embodiments of the invention, a concentration of fatty acids in a medium of the differentiation step is above 100 μM.

According to some embodiments of the invention, the priming step is 1-14 days long.

According to some embodiments of the invention, the intracellular fatty acid profile is as set forth in Tables 1 or 2.

According to some embodiments of the invention, a precursor thereof comprises acetyl-coA.

According to some embodiments of the invention, the precursor is selected from the group consisting of citrate, malonate, isocitrate and palamitic acid.

According to some embodiments of the invention, the culturing is effected in a serum-free medium.

According to an aspect of some embodiments of the present invention there is provided cells obtainable according to the method as described herein.

According to an aspect of some embodiments of the present invention there is provided cells comprising a genome of a first edible species and an intracellular fatty acid profile of a second edible species which is not of the first edible species.

According to an aspect of some embodiments of the present invention there is provided cells comprising a genome of a first edible species and an intracellular fatty acid profile of a second edible species which is not of the first edible species.

According to an aspect of some embodiments of the present invention there is provided a method of producing food, the method comprising combining the cells as described herein with an edible composition for human consumption.

According to an aspect of some embodiments of the present invention there is provided a food comprising the cells as described herein.

According to some embodiments of the invention, the food is devoid of animal components.

According to some embodiments of the invention, the food of the cells are non-GMO.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### IN THE DRAWINGS

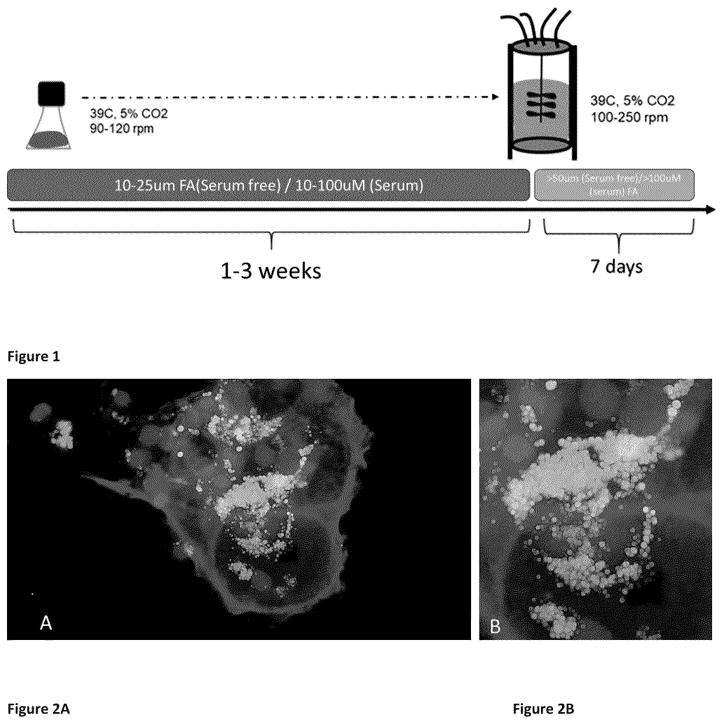

FIG. 1 is a schematic illustration showing a process for adipocyte priming and differentiation according to some embodiments of the invention.

FIGS. 2A-B show the differentiation of SM ES avian cell line into oleic acid lipid accumulating cells (adipocytes), A: phenotype of fully mature adipocytes originated from avian stem cells. Channels: green: BODIPY staining (lipids) red: Phalloidin (cytoskeleton) Blue: DAPI (nuclei). B: higher magnification of fat differentiated cell.

### DESCRIPTION OF SPECIFIC EMBODIMENTS OF THE INVENTION

The present invention, in some embodiments thereof, relates to methods of producing a food ingredient.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cultured meat provides a hope that society can become less reliant on animals for meat, thus reducing the environmental and health impact of animal farming.

However, it can be costly and time-consuming to develop multiple stem cell lines suitable for myriad of products. First, cell line engineering requires advanced gene delivery technology. It remains challenging to deliver genes into cells that confer desirable traits like fast biomass accumulation, Conventional methods using viruses introduce viral genes into the host genome, while bulk electroporation generally provides low transfection yield.

Whilst conceiving embodiments of the invention, the present inventors have devised a novel concept for improving or altering the organoleptic properties of cell lines used in the cultured meat industry. The present inventors have configured an approach which allows imparting xeno lipid profile to an established cell line thus combining the benefits of the cell line in terms of culturing and nutritional values, e.g., proteins with lipid profiles of interest, which allow for instance, imparting a fish organoleptic property in an avian cell line.

As shown in the Examples section, which follows, e.g., Examples 2-4, the exemplary protocols provided herein are based on priming stem cells at a low overall fatty acid concentration e.g., below 100 uM or below 25 uM (dependent on the presence or absence of serum, respectively) to adopt an adipocyte commitment and then differentiating the cells in the presence of a high fatty acid concentration (above 100 uM) so as to adopt a fatty acid profile of a target species, which is not that of the species of the stem cells.

Thus, according to an aspect of the invention, there is provided a method of producing a food ingredient, the method comprising:
(a) providing stem cells; and
(b) culturing said stem cells in the presence of an effective amount of fatty acids or precursor thereof selected to reach an intracellular fatty acid profile of an edible species of interest which is not of the stem cells or adipocytes differentiated therefrom, so asto result in a food ingredient having a lipid organoleptic property of said edible species.

As used herein “a food ingredient” refers to an edible ingredient preferably by human beings. However, the term is also meant to encompass feed ingredients which can be consumed by livestock. An ingredient can refer to a final food product or to an ingredient thereof, e.g., a nutritional supplement.

As used herein “edible” refers to a composition which is safe for human or animal eating. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

As used herein “food grade” refers to a substance which is either safe for human consumption or confirmed to come into direct contact with food products.

According to some embodiments of the invention, the materials used in the context of the invention are of “food grade” classification.

Such substances are also referred to herein and in the art as “food contact substances” or “food contact materials”.

The phrase “food contact substance” or FCS, is used herein to describe substances that are generally safe for human consumption by virtue of being generally recognized as safe (GRAS) or by passing standard safety tests, and thus qualify for use as a component of materials used in manufacturing, packing, packaging, transporting, or holding food, in the same manner it is meant in the guideline and regulation of worldwide food administration authorities, such as, for example, the U.S. Food and Drug Administration (FDA), Center for Food Safety and Applied Nutrition (CFSAN), the Office of food Additive Safety.

The phrase “generally recognized as safe” or GRAS, as used herein, is meant in the same manner which is defined, for example, under sections 201(s) and 409 of the U.S. FD&C Act. The U.S. law states that any substance that intentionally contacts food or added to food is a food additive, that is subject to premarket review and approval by FDA, unless the substance is generally recognized, among qualified experts, as having been adequately shown to be safe under the conditions of its intended use, or unless the use of the substance is otherwise excluded from the definition of a food additive. GRAS substances are distinguished from food additives by the type of information that supports the GRAS determination, that it is publicly available and generally accepted by the scientific community, but should be the same quantity and quality of information that would support the safety of a food additive. Since the qualification to an FCS or GRAS category can be obtained through a process of applying, testing and qualifying to the requirements of the various official food and drug authorities, the present embodiments are meant to encompass all relevant substances and their derivatives which are to become FCSs and GRAS in the future, as well as those which already qualify as FCSs and GRAS.

According to a specific embodiment, the food or food ingredient is nongenetically modified (GMO).

Being a food ingredient, the term does not relate to human, primate or murine/rat cells. As such their intracellular fatty acid profile is not an intended target according to some embodiments of the invention.

As used herein “stem cells” refers to cells which are not at their terminal differentiation state and hence are of sufficient proliferative capacity to allow expansion in culture. |Stem cells refers to undifferentiated or partially differentiated cells that can differentiate into at least one or various types of cxells and proliferate to produce more of the same stem cell.

According to a specific embodiment, the cells are of domesticated animals, such as listed below (e.g., duck, chicken).

According to a specific embodiment, the cells are produced by in vitro expansion e.g., stem cell expansion.

According to An alternative or an additional embodiment, the cells are produced by in vitro differentiation.

According to a specific embodiment, the stem cells are partially differentiated (e.g., blood-derived mesenchymal precursor cells, neural progenitor cells, multipotent adult progenitor cells, mesodermal progenitor cells, muscle progenitor cells).

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

According to a specific embodiment, the cells are multipotent stem cells are, i.e., cells that have the capacity to self-renew by dividing and to develop into multiple specialized cell types present in a specific tissue or organ. Most adult stem cells are multipotent stem cells.

According to a specific embodiment, the stem cells are adult stem cells (e.g., mesenchymal stem cells, multipotent stem cells, hematopoietic stem cells, liver-derived hematopoietic stem, marrow-derived stem cell, islet-cells producing stem cells, pancreatic-derived pluripotent islet-producing stem cells).

Hematopoietic stem cells, which may also be referred to as adult tissue stem cells, include stem cells obtained from blood or bone marrow tissue of an individual at any age or from cord blood of a newborn individual.

According to a specific embodiment, the adult stem cells are of the mesoderm lineage.

According to a specific embodiment, the adult stem cells are mesenchymal stem cells.

Mesenchymal stem cells are multipotent stromal cells that can differentiate into a variety of cell types, including osteoblasts (bone cells), chondrocytes (cartilage cells), myocytes (muscle cells) and adipocytes (fat cells which give rise to marrow adipose tissue). The term encompasses multipotent cells derived from the marrow as well as other non-marrow tissues, such as placenta, umbilical cord blood, adipose tissue, adult muscle, corneal stroma or the dental pulp of deciduous baby teeth. The cells do not have the capacity to reconstitute an entire organ.

As used herein “pluripotent stem cells” refers to non-human cells which can differentiate into all three embryonic germ layers, i.e., ectoderm, endoderm and mesoderm or remaining in an undifferentiated state. The pluripotent stem cells include embryonic stem cells (ESCs), e.g., naïve or primed, and induced pluripotent stem cells (iPS).

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, and cells originating from an unfertilized ova which are stimulated by parthenogenesis (parthenotes).

The main source for avian embryonic stem cells is a fertilized unincubated egg (Day 0). At this stage the embryo consists of 60-100K pluripotent cell locked in arrest state. The arrest phase is crucial in order to allow the hen to synchronize the hatching of several eggs that was being laid in different days. Propagation of these cells in-vitro occurs upon incubation in 39° C. (e.g., Pokharel, N et al. Poult Sci. 2017 Dec. 1; 96(12):4399-4408. doi: 10.3382/ps/pex242. PMID: 29053871).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell.

According to a specific embodiment, the stem cells are in single cell cultures, in such cultures, the cells are typically distinct but clumping to some extent may be present, whereby cell clumping does not exceed 29 um per clump.

Methods of growing and differentiating cells for the cultured meat industry are well known in the art. For example, WO2020/104650, WO2018/011805 and WO2020/123876 teach methods of growing stem cells and protocols for differentiation, each of which is incorporated herein in its entirety.

As mentioned, the cells are non-human pluripotent stem cells.

According to a specific embodiment, the stem cells are of livestock stem cells.

According to a specific embodiment, the stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

As used herein, the term “avian” to any species, subspecies or race of organism of the taxonomic Class Ayes, such as, but not limited to, such organisms as chicken, turkey, duck, goose, quail, pheasants, parrots, finches, hawks, crows and ratites including ostrich, emu and cassowary. The term includes the various known strains of Gallus gallus (chickens), for example, White Leghorn, Brown Leghorn, Barred-Rock, Sussex, New Hampshire, Rhode Island, Australorp, Cornish, Minorca, Amrox, California Gray, Italian Partidge-coloured, as well as strains of turkeys, pheasants, quails, duck, game hen, guinea fowl, squab, ostriches and other poultry commonly bred in commercial quantities.

In a specific embodiment, the avian cells are chicken cells.

In one example, the cells are from avian embryonic-derived stem cell line EB14 (chicken) or EB66 (duck) (WO2005042728).

According to an embodiment, the cells throughout the methods are non-genetically modified.

In one embodiment, the pluripotent stem cells are of a stem cell line.

Pluripotent stem cells are adherent by nature and hence are grown under conditions of cells adherence, also referred to as a two dimensional culture (2D).

According to specific embodiments, a 2D culture relates to growth on a two-dimensional matrix (feeder layer-free) or on feeder cells.

Thus, the pluripotent stem cells can be grown (expanded) on a solid surface such as an extracellular matrix (e.g., gelatin, fibronectin, Matrigel® or laminin) in the presence of a culture medium.

According to a specific embodiment, the surface is gelatin.

According to a specific embodiment, the cells are grown on a feeder layer.

According to a specific embodiment, the feeder cells are mouse embryonic fibroblasts (MEFS).

According to another embodiment, the stem cells are cell aggregates.

WO2022/149142 teaches specific embodiments for obtaining aggregates and is hereby incorporated by reference in its entirety.

As used herein “an aggregate” refers to a group of proliferative, non-differentiated i.e., pluripotent cells that are bound to each other via secretion of adhesive molecules (e.g. ECM). The size can be between 30-1000 um e.g., 50-500 um, e.g., 30-100 um, 100-500 um, 100-400 um, 200-500 um, 300-400 um, 100-200 um, 50-200 um, 500-1000 um, 700-1000 um. It will be appreciated that where size is indicated throughout the document, the size refers to an average size in a population of aggregates.

Markers for pluripotency typically include Oct4+, Lin28+, SSEA1+, SSEA4−, ENS1+, Tra-1-60+. nanog+. According to a specific embodiment, the markers are: Oct4+, Lin28+, SSEA4-, ENS1+, Tra-1-60+. Nanog+.

According to a specific embodiment, the aggregate is grown in the presence of growth factors (e.g., IGF1, SCF, IL6, IL6Rα, LIF hLIF combinations thereof, or additionally or alternatively, IWR1, FGF2 or others) or signaling inhibitors such as inhibitors of Rho (e.g., Y27632) MEK, GSK3, FGFR3, N2B27-3, or as further exemplified below.

According to a specific embodiment, about 90-100% of the cells in the aggregates are pluripotent stem cells.

The following is an exemplary protocol for generating the aggregates.

According to a specific embodiment, pluripotent stem cells, e.g., embryonic stem cells, are cultured in the presence of factors and optionally serum (e.g., fetal bovine serum, horse serum, and/or fish serum from trout) or serum replacement (e.g., yeast or plant hydrolysates e.g., soy. Other factors may be included at this stage e.g., Na-pyruvate, Na-selenite, amino acids, 2-mercaptoethanol. Cells are allowed to propagate and passaged every 24-72 hrs.

According to a specific embodiment, the growth factor is selected from the group consisting of IGF-1, IL6, sIL6 Rα, hLIF and stem cell factor (SCF).

According to a specific embodiment, the growth factors comprise IGF-1, IL6, sIL6 Rα, hLIF and stem cell factor (SCF).

According to some embodiments of the invention, when the cells reach 3-5 passages they are gradually deprived from substance adherence (e.g., feeder layer) and grown for several passages to select for stable feeder-free clones. According to some embodiments, factors are still present at this stage.

Notably, after the feeder layer withdrawal phase, the cells tend to form less compact stem cell colonies composed of large nucleated cells as they are not constrained by fibrous cells. According to a specific embodiment, at this stage, cells exhibit the expected doubling time of about 24 hours per cycle. This stage is also referred to as “gradually depriving the non-human stem cell line of the matrix adherence”.

As used herein “gradually depriving” refers to deprivation from matrix adherence (not from GFs).

To do this, cells are gradually adapted to grow in suspension rather than as adherent cells.

Generally, stem cells which are contemplated herein, single cells or aggregates, can be grown in adherent conditions (2D or 3D) or in a suspension culture.

As used herein the phrase “suspension culture” refers to a culture in which the pluripotent stem cells are suspended in a medium rather than adhering to a surface.

Thus, the culture of the present invention is “devoid of matrix adherence” in which the pluripotent stem cells are capable of expanding without adherence to an external substrate such as components of extracellular matrix, a glass microcarrier or beads.

According to some embodiments, as taught in WO2022/149142 cells are gradually displaced from adhesive surfaces (such as those comprising an adhesive matrix e.g., gelatin, laminin, fibronectin, poly-L-lysine) and subtle shaking is imposed (e.g., 50-100 rpm) and optionally mechanical dissociation of the aggregates. Shaking may be gradually increased at every passage. According to a specific embodiment, by the continuous selection for a period of about 2-3 months, cells are encouraged to down-regulate different adhesion molecules while expressing others, allowing over time the formation of 3D loose raspberry-like aggregates, with a clear definition of each cell composing the aggregate, as opposed to a structure of an embryoid body. At this stage, cells have become very stable, and doubling time is reduced to between 10-20, e.g., 18-20 h, 10-12, h, 12-14 h, 12-16 h, 10-16 h, 12-18 h per cycle such as in the case of avian embryonic stem cells.

According to a specific embodiment, the aggregates are of an aggregate forming cell line. Thus, following the adaptation of cells to growth in suspension, the cells are adapted to continued rapid growth in a reproducible manner such as in a stirred bioreactor environment to ensure the ability of the cells to be suitable for industrial scale-up. For this, clones are tested for the generation of a cell line that grows as aggregated cells, with a high proliferative rate, optionally in high-velocity stirring (200-400 rpm tip speed) in stirred bioreactors, while maintaining the aggregate's integrity and stem cell characteristics. Following this process of optimization, several cell lines are produced with all favorable characteristics, these cells are named “SMCMC” or “SM-ES” (a chicken line, see Examples section), according to some embodiments of the invention. These cells exhibit the desired morphology, differentiation potential, and a doubling time of 10-12 hours per cycle, with some growing conditions showing 8 hours per cycle.

Such aggregate forming cell lines can be stored in a cell bank.

According to an aspect of the invention there are provided pluripotent stem cell aggregates.

According to a specific embodiment, the aggregates are obtainable according to the methods as described herein.

According to a specific embodiment, the aggregates exhibit an average diameter of 80-120 pm.

According to a specific embodiment, the non-human pluripotent stem cells of the aggregates exhibit alkaline phosphatase expression.

According to a specific embodiment, the non-human pluripotent stem cells of the aggregates exhibit telomerase gene expression.

According to a specific embodiment, the non-human pluripotent stem cells of the aggregates are SSEA4−, LIN28+, ENS-1+, NANOG+, OCT4, + and TRA-I-60+, such as determined at the RNA level.

According to a specific embodiment, the hon-human pluripotent stem cells of the aggregates do not display oncogenic transformation.

According to an aspect of the invention there is provided a pluripotent stem cell aggregate comprising non-human pluripotent stem cells, the non-human pluripotent stem cells of the aggregates exhibiting a doubling time of no more than 12 hours in an undifferentiated manner for more than 60 passages, capable of differentiating into muscle, fat and connective tissue upon differentiation induction and exhibiting cell to cell adhesion lower than that of embryoid bodies (EBs) as determined by reduced expression of adhesion molecules selected from the group consisting of COL6A2, CD44, COL6A1, ANXA1, ANXA2 and S100A11 as compared to the EBs.

According to a specific embodiment, the pluripotent stem cell aggregate exhibits at least one of:
(i) an average diameter of 80-120 um;
(ii) the non-human pluripotent stem cells of the aggregate exhibit alkaline phosphatase expression;
(iii) the non-human pluripotent stem cells of the aggregate exhibit telomerase gene expression; (iv) the non-human pluripotent stem cells of the aggregate are SSEA4−, LIN28+, ENS-1+, NANOG+, OCT4, + and TRA-1-60+;
(v) exhibit organoleptic properties of a native meat product;
(vi) the aggregates exhibit about the same gene expression as that of a stem cell line from which they are derived, excluding expression levels of cell motility and migration-related genes.

According to a specific embodiment, the aggregate exhibits a combination of i+ii. I+ii+iii, i-iv, i-v, i-vi, ii-iii, ii-iv, ii-v, ii-vi, iii-iv, iii-v, iii-vi, iv-v, iv-vi, V-vi.

According to a specific embodiment, the non-human pluripotent stem cells are selected from the group of avian pluripotent stem cells, bovine pluripotent stem cells, porcine pluripotent stem cells, goat pluripotent stem cells, sheep pluripotent stem cells, shrimp pluripotent stem cells and fish pluripotent stem cells.

According to a specific embodiment, the avian pluripotent stem cells are selected from the group of chicken pluripotent stem cells and duck pluripotent stem cells.

According to a specific embodiment, the avian pluripotent stem cells are chicken pluripotent stem cells.

According to a specific embodiment, the aggregates exhibit about the same gene expression as that of a stem cell line from which they are derived, excluding expression levels of cell motility and migration-related genes, such as determined at the RNA level (see Examples section) or protein level (e.g., immunostaining).

According to a specific embodiment, the cells of the aggregates keep a normal karyotype.

According to a specific embodiment, the above covers steps 1-7 of FIG. 19 of WO2022/149142. According to a specific embodiment these steps are performed in the presence of serum, although, as mentioned, serum can be replaced by a serum replacement or other substitutes such as yeast or plant hydrolysates.

In order to achieve growth in the absence of serum, post formed aggregates (in shaking conditions) are adapted to grow in serum free media.

The adaptation to serum free media takes place following the propagation of ES cells as aggregates in shaking flasks (FIG. 19 of WO2022/149142, stage 7b) either by additional propagation in flasks using serum free medium (FIG. 19 of WO2022/149142, stage 7b) and then transferring to stirred bioreactor (FIG. 19 of WO2022/149142 stage 8b) or by direct seeding in bioreactor using serum free media (FIG. 3, stage 8b). The adaptation to serum free media is done by collecting highly proliferative aggregates populations and introducing them to serum—free media (FIG. 19 of WO2022/149142, 7b). Gradual adaptation is carried out by reducing the percentage of serum e.g., FBS within the serum free media over time (e.g., 5% to 2.5% to 1% until complete withdrawal). In most cases the gradual adaptation duration is between 2-4 weeks. Another possibility to generate serum-free (SF) culture for high scale production is to transfer the aggregates directly to the serum free media. In this procedure, cells with the ability to survive and quickly adapt to the changed environment are collected. The length of such process is about 2 weeks. Either possibilities is concluded by the generation of cell bank for future use (FIG. 19 of WO2022/149142 step 8a), according to some embodiments of the invention.

Cells can grow in other combinations of basal media supplemented with yeast, plant peptones and hydrolysates. Among the mixes which were used successfully for adaptation of cells to serum-free media, the present inventors could identify: DMEM (high glucose) supplemented with the Ex-Cell lysate, DMEM/F12 supplemented with Ex-Cell lysates, DMEM (low glucose) supplemented with Ex-Cell lysate, DMEM (high glucose) supplemented with combination of soy and yeast lysates manufactured by KERRY group. Lysates that tested successfully in this process were either a combination of all or part of these four products as follows: Hypep 1510 (ID: S-2048780, Item: U1-5X99023), SHEFF-VAX PLUS ACF(ID S-2048778, U1-5X00484.K1G), SHEFF-VAX PF ACF (ID:S-2048777, U1-5X01143.K1G), SHEFF-VAX PLUS PF ACF VP (ID: S-2048776, U1-5X01090).

As mentioned, the cells (single cells or aggregates) can be grown in suspension or under adherent conditions, in 2D or 3D settings. When a carrier or scaffold is employed measures are taken to use edible materials or protocols for removal of same prior to avoid inclusion in food.

Regardless of whether single cells or aggregates are employed, culturing the stem cells with a particular combination of fatty acids (which is different than that typically used for growing/expanding stem cells or aggregates e.g., basal-medium (DMEM/F12) containing amongst other things known in the art typically not more than 10 nM of linoleic and/or lipoic acid) is done to achieve an intracellular fatty acid profile of an edible species of interest which is not of the stem cells or adipocyte derived therefrom. In other words, the fatty acid profile has a xeno relationship with that of the stem cells i.e., the fatty acid profile is distinctive of that of the stem cells

It may be necessary to determine the fatty acid profile in the cells before culturing them with fatty acids in order to determine the exact recipe of fatty acids.

The fatty acids or precursors thereof are exogenous to the stem cells, i.e., they are added to the medium which is contacted with the cells.

Methods of determining fatty acids in cells are well known in the art. Methods of analyzing lipids and lipidomes include, but are not limited to, gas chromatography (GC), mass spectrometry (MS), and nuclear magnetic resonance (NMR) spectroscopy.

Hence, the target fatty acid profile is of an adipocye of a species which is not that of the stem cells.

Alternatively the target FA profile is an average profile of an edible tissue (e.g., chicken breast, bovine fillet etc.).

As used “fatty acid profile is of an adipocyte of a species” refers to the level and/or presence of the four major fatty acids i.e., oleic acid (OA), linoleic acid (LA), stearic acid (SA) and palmitic acid (PA). In the case of fish, this term also refers to those fatty acids which are absent from terrestrial species, i.e., DHA and EPA.

Alternatively the target FA profile is an average FA values of an edible tissue (e.g., chicken breast, bovine fillet etc.).

The target profile is meant to reach about the same of that of the species of the species of interest with respect to the above-mentioned FAs+/−10%.

As used herein “profile” refers to the type and level of fatty acids (FAs), which characterize the adipocyte of the species with respect to oleic acid, linoleic acid, stearic acid and palmitic acid. In the case of fish, this term also refers to those fatty acids which are absent from terrestrial species. Typically, the term profile refers to more than 1 (i.e., 2, 3, 4, 5, 6, 7, 8, 9, 10) fatty acid. i.e., at least 2, 3, 4, 5, 6, 7, 8, 9, 10, e.g., 4-8, 4-7,4-6.

Thus, for instance, the stem cell can be an avian stem cell and the fatty acid profile is bovine; the stem cell can be an avian stem cell and the fatty acid profile is fish; the stem cell can be an avian stem cell and the fatty acid profile is pork; the stem cell can be an avian stem cell and the fatty acid profile is of another avian species (e.g., chicken and duck, respectively); the stem cell can be a bovine stem cell and the fatty acid profile is fish; the stem cell can be a bovine stem cell and the fatty acid profile is avian; the stem cell can be a bovine stem cell and the fatty acid profile is pork; the stem cell can be a pork stem cell and the fatty acid profile is bovine, the stem cell can be a pork stem cell and the fatty acid profile is fish; the stem cell can be a pork stem cell and the fatty acid profile is avian. Although not specifically mentioned, other domesticated livestock are also contemplated herein such as ostrich and horse.

As used herein “to reach an intracellular fatty acid profile of an edible species” refers to culture conditions which modify the intracellular profile of the fatty acids in the cells during the differentiation stage such that it is not that of the cells (e.g., adipocytes) of the species the stem cells are derived from in terms of presence and/or level.

According to some embodiments, the modification is such that the fatty acid (FA) introduced into the culture is not present at all in the stem cells of the species e.g., long fatty acids (e.g., above C18, such as DHA). Hence, for instance, turning non-fish stem cells into fish like can be done by the inclusion of the FAs in Table 3 or Table 7 (each of which is considered a part of the present specification). For instance, according to an exemplary protocol, for turning the FA profile of chicken to tuna a direct differentiation protocol is employed (without priming): Docosahexaenoic acid (DHA), Eicosatetraenoic acid (EPA) and optionally icosapentaenoic acid can be included and incubated with the cells for 5-10 days, e.g., 7 days.

In other embodiments, a level of a specific FA can be elevated, such as by more than 1.5, 2, 5,10 folds with respect to the stem cells. For instance, to render an avian stem cell mature into a beefy tasting edible cell, linoleic can be added to the culture.

In order to produce a porcine FA profile, linoleic acid is to be increased while maintaining the relative amount of oleic acid. In order to produce a bovine FA profile the level of linoleic acid is to be increased at the expense of the relative amount of oleic acid.

As used herein “maintaining” or “retaining” or any grammatical deviation thereof refers to unchanging the relative level (+/−10%) of a specific fatty acid as that found in the stem cells or a cell of the species from which the cells are derived in nature. For instance, if a species comprises a 1:1 ratio in certain fatty acids (e.g., palmitic and linoleic) and this ratio is desired even in the target species, the medium will include equal levels of these FAs albeit their amount can change.

The conditions can be the inclusion of one or more (e.g., 2, 2-20, 3-20, 4-20, 5-20, 5-15, 3-15, 4-15, 5-12, 2-10, 3-10, 4-10, 5-10, 2-9, 3-9, 4-9, 5-9, 4-8, 4-7, 4-6, 2-5, 3-5, 4-5, 3-6) fatty acid or precursor thereof. However, generally the profile is governed by at least one of the four FAs, OA, PA, LA, and SA.

Thus, for instance, the species of interest can be aquatic (vertebrate or invertebrate) and the stem cells are avian (e.g., chicken or duck), the species of interest can be porcine and the stem cells are avian (e.g., chicken or duck); the species of interest can be bovine (e.g., cow, sheep, goat) and the stem cells are avian (e.g., chicken or duck).

When the stem cells already have an adequate level or higher of a fatty acid of the edible species of interest there is no need to add it to the culture such that its relative level is increased over other FAs in the culture only to an amount which retain its relative level in the cell (the numbers indicated herein and in the Tables relate to % w/w).

### Fatty Acid Profile in Different Species

The fatty acid profile of different species is well known in the art.

Exemplary fatty acid profiles of different species are provided below. Information is provided regarding several fish species (Table 1) and livestock (Table 2). Profiles of other edible species is available from the prior art.

TABLE 1
fatty acid profile of different fish species (%)

| | | | | | | | Alaska | |
|---|---|---|---|---|---|---|---|---|
| FA | Name | Sardine | Tuna | salmon | Shrimp | Tilapia | pollock | pangasius |
| C16:0 | palmitic | 16.2 | 22.7 | 11.86 | 19.18 | 23.54 | 25.34 | 24.08 |
| C16:1 | palmitoleic | 11.3 | 3.4 | 1.86 | 6.76 | 4.59 | 11.77 | |
| C18:0 | stearic | 1.3 | 9.5 | 3.06 | 10.62 | 7.83 | 5.67 | 6.6 |
| C18:1 | oleic | 9.8 | 13.7 | 44.59 | 13.64 | 32.21 | 21.68 | 21.73 |
| C18:2 | linoleic | 4.3 | 0.8 | 14.84 | 7.25 | 13.44 | 1.28 | 7.09 |
| C20:1 | gondoic | 2.6 | 1.2 | 2.79 | 1.4 | | | |
| C20:4 | arachidonic | 0.2 | | 0.31 | 4.67 | 2.71 | 0.38 | |
| C20:5 | EPA | 24.2 | 7.8 | 1.55 | 12.4 | | 14.79 | 2.45 |
| C22:1 | erucic | 4.8 | 3.6 | 9.8 | | 3.61 | | |
| C22:6 | DHA | 6.5 | 32.5 | 3.94 | 8.8 | | 10.03 | 0.23 |

TABLE 2
fatty acid profile of representative livestock animals (%)

| | | | | | SM ES |
|---|---|---|---|---|---|
| | | | | | (proprietary |
| | | | | | chicken |
| Name | FA | LAMB | BOVINE | PORK | ES line) |
| Palmitic acid | C16:0 | 27.3 | 25.9 | 24.96 | 25.1 |
| Palmitoleic acid | C16:1 | 1.64 | 0.9 | 2.2 | 6.2 |
| Stearic acid | c18:0 | 13.7 | 14.5 | 13.86 | 14.6 |
| oleic acid | c18:1 | 28.9 | 33.4 | 43.92 | 41.7 |
| linoleic acid | c18:2 | 7.77 | 4.13 | 12.64 | 0.5 |

### REFERENCES

Lamb-PLoS ONE 11(6): e0156765.
www(dot)doidotorg/10dot1371/journal(dot)ponedotO156765

Bovine-IntJ Biol Sci 2012; 8(2):214-227. doi: 10.7150/ijbs. 8.214

Pork-Meat Science Volume 76, Issue 1, May 2007, Pages 54-60

### A Non Limiting List of Specific Protocols is Provided Below:

Generation of fish-like fatty acid profile (Examples: Tuna, Salmon, shrimp, Tilapia, Alaska Pollock, Pangasius).

In order to allow the generation of cultivated avian cells harboring fatty acid profiles that exist in different fish species, the following exemplary protocols can be used. Generally, protocols include the combination of fatty acids needed to mimic or direct fatty acid profile into the one that exists in the edible species of interest. Such combinations are presented in Table 3, where the stem cells modified are of any non human source e.g., avian e.g., primary (embryonic day 0) or established ES cell lines e.g., SM-ES, embryonic mesenchymal stem cells (E3-E21) or adult mesenchymal stem cells.

TABLE 3
(values are shown in % and provided for the differentiation step)

| | | | | | | | Alaska | |
|---|---|---|---|---|---|---|---|---|
| FA | Name | Sardine | Tuna | salmon | Shrimp | Tilapia | pollock | pangasius |
| C16:0 | Palmitic | | | 10-500 um | | | | |
| C16:1 | palmitoleic | 10-500 um | 10-500 um | | | | 10-500 um | |
| C18:0 | Stearic | | 10-500 um | | | | | |
| C18:1 | Oleic | | | | | | | |
| C18:2 | Linoleic | 10-500 um | | 10-500 um | 10-500 um | 10-500 um | 10-500 um | 10-500 um |
| C20:1 | Gondoic | | | 10-500 um | | | | |
| C20:4 | arachidonic | | | | 10-500 um | 10-500 um | | |
| C20:5 | EPA | 10-500 um | 10-50r0 um | 10-500 um | 10-500 um | | 10-500 um | 10-500 um |
| C22:1 | Erucic | 10-500 um | 10-500 um | 10-500 um | | 10-500 um | | |
| C22:6 | DHA | 10-500 um | 10-500 um | 10-500 um | 10-500 um | | 10-500 um | 10-500 um |

### Generation of Bovine/Lamb/Pork-Like Fatty Acid Profile in Avian ES Cells

Protocols designed to mimic fatty acid profiles of other livestock species are provided below in Table 4 which represent some embodiments of the invention.

TABLE 4
(values are shown in % and provided for the differentiation step)

| FA | Name | LAMB | | BOVINE | Pork |
|---|---|---|---|---|---|
| Palmitic acid | C16:0 | 10-500 | uM | 10-500 uM | 10-500 uM |
| Palmitoleic acid | C16:1 | | | | |
| Stearic acid | c18:0 | 10-500 | um | | 10-500 uM |
| oleic acid | c18:1 | 10-500 | uM | 10-500 uM | |
| linoleic acid | c18:2 | 10-500 | uM | 10-500 uM | 10-500 uM |

Exemplary levels of FAs in stem cells of chicken is provided above in Table 2.

### Differentiation Process:

To achieve various fatty acid profiles, a direct differentiation protocol (as in Example 1 below) is applied or a step-wise procedure.

As used herein “direct differentiation protocol” refers to the addition of FAs at high concentrations to a stem cell culture e.g., pluripotent stem cells, (above 25 uM or above 100 uM, when serum is absent or present in the culture) stem cells in order to allow the cells to accumulate fat. This process does not include a priming step.

As used herein “step-wise differentiation protocol” refers to a process of culturing the stem cells in a priming medium (with an overall fatty acid concentration of below 25 uM or below 100 uM, when serum is absent or present in the culture) to adopt a pre-adipocytic identity and following applying a differentiation protocol in the presence of above 25 uM or above 100 uM, when serum is absent or present in the culture.

According to a specific embodiment, the differentiation includes “priming” cells to undergo pre-adipocyte differentiation followed by “differentiation” process, as exemplified in FIG. 1. Such a protocol can be considered a step-wise differentiation protocol.

Priming: refers to directing the stem cells to a mesodermal lineage e.g., pre-adipocyte stage. According to some embodiments, the cells retain their proliferative properties for at least 60 passages in culture. Markers for proliferating stem cells include, but are not limited to, Oct4, Nanog, SSEA1 (optional), ENS1. Markers for mesenchymal stem cells include, but are not limited to, Stro-1 and CD90.

That step includes the addition of FA in low doses (below 25 uM or below 100 uM, when serum is absent or present in the culture) to direct cell to an adipocyte lineage.

Differentiation: the step that includes administration of FAs in high doses (above 100 uM e.g., 100-500 uM) to enhance terminal differentiation into adipocytes.

Thus, the priming step includes adjusting the (e.g., avaian) stem cells to growth in the presence of low dosages of different fatty acids. In this process, the present inventors add a certain or several fatty acids to the cell's media at a final concentration that does not exceed in overall 25 uM (e.g., in the absence of serum, 10-25 uM or 10-20) or does not exceed 100 uM in the presence of serum (e.g., or 10-100 uM, 10-50 uM). The growth under these conditions should proceed for 1-14 days, e.g., 1-10 days, 1-12 days, 5-14 days, 7-14 days.

In the priming step the cells are typically treated with transferrin and insulin without proliferation factors while in the presence of low level of FAs, typically below 100 uM or below 25 uM as a function of serum presence (lower levels in the absence of serum). Examples include but are not limited 0.1-90 uM, 0.1-80 uM, 0.1-70 uM, 0.1-60 uM, 0.1-50 uM, 0.1-40 uM, 0.1-30 uM, or 0.1-25 mM, 0.1-20 mM, 0.1-15 mM, 0.1-10 mM, 0.1-5 mM, 5-30 uM, 5-25 uM, 5-20 uM, 5-15 uM, 5-10 uM.

In the differentiation step (other factors are still present such as insulin and transferrin), priming cells to adipogenc differentiation is followed by a differentiation procedure. The step of differentiating cells includes elevating the levels of FA, above 100 uM (in the presence of serum), e.g., to 500 uM, e.g., for up to 14 days, e.g., 1-7 days, 1-10 days, 5-10 days; or above 30 uM (the cutoff changes when serum is absent), e.g., to 500 uM, e.g., 35, 30-500 uM, 50-500 uM, 100-500 uM, 150-500 uM, 200-500 uM, 300-500 uM, 400-500 uM, 50-400 uM, 150-400 uM, 200-400 uM, 250-400 uM, 300-400 uM, 350-400 uM, 120-300 uM, 150-300 uM, 200-300 uM, 250-300 uM, e.g., for up to 14 days, e.g., 1-3 days, 1-7 days, 1-10 days, 5-10 days.

The resultant cells are endowed with unique organoleptic properties.

As used herein the term “organoleptic properties” refers to the aspects of food, that a consumer experiences via the senses-including taste, sight, smell or touch.

For example, adipocytes may be used to confer a non-meat food with a meaty taste and/or texture (e.g., beef or chicken) when cooked or grilled.

Methods of organoleptic assaying are well known in the art, some of which are described infra. It makes use of the senses to evaluate the general acceptability and quality attributes of the products. The assays typically make use of dedicated panelists and/or artificial means.

In addition, the resultant cells confer nutritional values by virtue of the presence of a unique fatty acid profile in the cells to which they were added.

WO2018/189738 describes assays for analyzing organoleptic properties of foods and is hereby incorporated by reference in its entirety.

Thus, the present teachings also contemplate the cells obtainable according to the present teachings.

Alternatively or additionally, there is provided cells comprising a genome of a first edible species and an intracellular fatty acid profile of a second edible species which is not of the first edible species.

Thus, the cells comprise may comprise a transcriptome and/or a proteome of the first edible species (e.g., an adipocyte of the first edible species) but not the fatty acid profile characteristic of the cell of the first species but rather of a second edible species.

These can be used in the food industry.

Thus, according to an aspect there is provided a method of producing food, the method comprising combining the cells with an edible composition (which is not derived from meat, i.e., non-meat) for consumption, e.g., by humans.

According to a specific embodiment, the non-meat is a plant originated substance(s).

According to a specific embodiment, the non-meat is a non-plant originated substances [e.g., minerals, synthetic substance(s)].

According to a specific embodiment, the non-meat is selected from the group consisting of a plant originated substance(s) and non-plant-originated substance(s).

According to a specific embodiment, the foodstuff is a vegetarian foodstuff.

According to a specific embodiment, the foodstuff is a vegan foodstuff.

According to a specific embodiment, the foodstuff comprises a meat substitute or is generally consumed as a meat substitute (plant-based).

According to a specific embodiment, the animal cells are of a single cell type or single cell lineage.

As used herein “cell lineage” refers to any of endoderm, mesoderm and endoderm.

According to a specific embodiment, the animal cells are of no more than two cell types or two cell lineages.

According to a specific embodiment, the animal cells are of no more than three or four cell types.

According to a specific embodiment, the foodstuff is free of bodily fluids e.g., saliva, serum, plasma, mucus, urine, feces, tears, milk etc. As used herein “animal cells” refer to “non-human cells”.

According to a specific embodiment, the term food also encompasses an ingredient used in preparing a final product.

According to an embodiment of the invention the food is an end article of manufacture (product) to be consumed by a human or non-human subject.

Examples of meat substitutes: natural, traditional and commercially made which are contemplated according to some embodiments of the invention:
Alpro and Provamel, both usually known for their plant milk range, also offer different vegetarian meat substitutes
Beanfeast
Beyond Meat
Boca Burger
Falafel, a traditional Middle Eastern bean fritter, believed to have been created by ancient Copts as a meat substitute during Lent
Fistulina hepatica, common mushroom known as beefsteak fungus
Ganmodoki, a traditional Japanese tofu based dish similar to veggie burgers
Gardein
Gardenburger
Glamorgan sausage
Goshen Alimentos
Green Slice vegetarian, organic and soy free hot dogs and deli slices
Impossible Foods
Jackfruit, a fruit whose flesh has a similar texture to pulled pork when cooked
Koya-dofu, freeze-dried tofu that has a taste and texture similar to meat when prepared, common in
Buddhist vegetarian cuisine
Laetiporus, a mushroom which is also named chicken of the woods
Leaf protein concentrate
LightLife
Linda McCartney Foods
Lyophyllum decastes, mushroom known as fried chicken mushroom
Meat extenders
Meatless
Mock duck
Morningstar Farms
Muscolo di grano (Wheat's muscle), seitan prepared according to an Italian recipe
Nut roast
Oncom
Paneer, for example in such dishes as Paneer tikka
Quorn
Soy protein
Soy pulp, used for veggie burgers and croquettes
Tempeh
Textured vegetable protein
Tofu, not traditionally seen as a meat substitute in Asia, but widely used for that purpose in the
Western hemisphere
Tofurkey
Tofurky
Turtle Island Foods
Vegetarian bacon
Vegetarian hot dog
Veggie burger
Veggie Chicken patty
Veggie Chicken cutlet, breaded (schnitzel)
Veggie Chicken frankfurter (Hot dog)
Viana, one of the largest German vegan food manufacturers, offers a wide range of veggie burgers, croquettes, sausages, minced mock meats, up to vegan döner, vegan gyros and deli slices for sandwiches
Wheat gluten

Any of the above examples is an independent embodiment that is not necessarily associated with a specific vendor.

As mentioned, the food preparation is performed by combining a plant-originated substance with an amount of cultured meat cells.

The terms “connection between” and “interconnected with” as defined above refers to those specific cases where at least two different portions are provided inter alia, by any suitable means, including (i) where the at least one second portion is comprised, immersed, wetted, solubilized, suspended, doped, glued, attached, aggregated, mixed, contained or otherwise, directly or indirectly, provided in contact within and/or upon or on top of the aforesaid at least one first portion; and (ii), where the at least one second portion is chemically, biologically and/or physically reacts with the aforesaid at least one first portion so that either integral or non-integral non-cultured meat and cultured meat substantially unified phase, matter or composition is obtained.

The combined matter can be subjected to further processing such as by means of rising, kneading, extruding, molding, shaping, cooking, stewing, boiling, broiling, baking, frying and any combination of same.

According to a specific embodiment, the % values indicated herein (or other values such as timing) are about those indicated.

As used herein the term “about” refers to ±20 or 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

The Fatty Acid Profile of Avian Pluripotent Stem Cells and its Manipulation Towards that of a Chicken or Tuna Cells

A stem cell line or in an embodiment of the invention “the SM-ES” (SuperMeat Culture meat cells) refers to a cell line of embryonic stem cells of avian origin, that can be in the form of isolated cells or aggregates as long as it contains stem cells. Embryonic stem cells can be driven to undergo cellular differentiation to all three lineages.

### Materials and Methods

Cells: cells used in the following examples originated from a proprietary line termed “SM” which are chicken established embryonic stem cells generated from a day 0 (un-incubated) fertilized egg. Cells were propagated in culture after removal from the egg to a plates in a DMEM F12 (Gibco #11320033)/10% FBS (Gibco #26140079) medium supplemented with 5 ng/L h-LIF (Peprotech #300-05).

Fatty acid analysis: analysis of fatty acids was done using HPLC-based methods. Analysis was performed using outsourced services (Interdepartmental Equipment Unit, HUJI).

Phalloidin staining: for cytoskeleton staining, cells were fixed using 4% PFA and permeabilized using PBS/0.01% triton. Staining was performed by the addition of phalloidin-conjugate to iFlour555 (Abcam #abl76756) for 90 minutes. Following staining, cells were washed with PBS for three times and taken for microscopic analysis (Olympus IX71).

Lipids staining: lipids staining was performed using BODIPY (Thermo-Fisher D3922) at a concentration of 1:1000.

Generating a Tuna-like profile in avian stem cells: For preparation of a Tuna-like fatty acid profile: a population of Avian embryonic stem cells (SM ES) were propagated as pluripotent cells in ES medium at a concentration of 0.3M cells/ml in flasks. Upon reaching 10×10⁶ cells/well, the cells were transferred to stirred bioreactor for further propagation in complete medium until reaching 3-4×10⁶ cells/ml. Following propagation, the medium was changed to a differentiation medium (100 um OA, DHA, EPA). Cells were harvested following 7 days and subjected to fatty acid analysis.

ES medium composition: DMEM/F-12, 10% Fetal Bovine Serum, 1X MEM Non Essential Amino Acid concentrate, 1 mM Sodium Pyruvate, 10 U/1 U Penicillin/Streptomycin, 2 mM glutamine, 15 uM β-Mercaptoethanol, 5 ng/mL IGF1, 1 ng/mL SCF, 1 ng/mL IL6, 1 ng/mL sIL6 Ra, 20 ng/mL (1,000 U/mL) hLIF.

### Results

As a first step, the present inventors conducted a thorough analysis of fatty acid/lipids profiles on the SM-ES cells. In this analysis, the present inventors included samples taken from conventionally produced chicken breast tissue. The profiles are shown in Table 5 below.

TABLE 5
fatty acid profile of chicken breast tissue (literature “Ross”
and fresh tissue test “Breast”) as well as the proprietary cell line SM-ES.

| FA | Name | ROSS* | SM ES |
|---|---|---|---|
| C16:0 | palmitic | 23.65 | 25.1 |
| C16:1 | palmitoleic | 6.48 | 6.2 |
| C18:0 | stearic | 6.89 | 14.6 |
| C18:1 | oleic | 38.80 | 41.7 |
| C18:2 | linoleic | 19.39 | 0.5 |
| C20:1 | gondoic | 0.0 | 2.4 |
| C20:4 | arachidonic | 1.89 | 1 |
| C22:6 | cervonic | 0.0 | 0.82 |

The effects of feeding distillers dried grains with solubles on broiler meat quality. Poultry science. 88. 432-9. 10.3382/ps.2008-00406.

Results are able to demonstrate the similarity in the profile of the SM ES cells and the values got from fresh tissue analysis. In addition, the values correlate with literature data regarding the commercial broiler breed Ross.

However, since linoleic acid adds a beefy taste, the present inventors didn't want to elevate its concentration.

Next step was to test the ability to control the fatty acid profile of SM ES cells by growing the cells in the presence of certain or several fatty acids (FAs), dependent on the selected edible species. In these experiments, the present inventors demonstrated the ability to double the proportion of oleic acid (OA) within cells by culturing in the presence of 300 uM using a direct differentiation protocol. The full results of these analyses are summarized in the Table 6 below.

TABLE 6

| | | O.A + DHA + EPA |
|---|---|---|
| Fatty Acid | | 100 uM each |
| Lauric acid | C12:0 | 0.00 |
| Myristic acid | C14:0 | 0.00 |
| Myristoleic acid | C14:1 (cis-9) | 0.00 |
| Pentadecanoic | C15:0 | 0.00 |
| acid | | |
| Palmitic acid | C16:0 | 0.60 |
| Palmitoleic acid | C16:1 (cis-9) | 2.04 |
| Stearic acid | C18:0 | 7.83 |
| | C18:1 (trans-9) | 0.00 |
| Oleic acid | C18:1 (cis-9) | 18.06 |
| | C18:1 (cis-11) | 2.04 |
| | C18:2 | 0.00 |
| | (trans-9, 12) | |
| Linoleic acid | C18:2 | 0.00 |
| | (cis-9, 12) | |
| g-linoleic acid | C18:3 | 0.00 |
| | (cis-6, 9, 12) | |
| a-linoleic acid | C18:3 | 2.08 |
| | (cis-9, 12, 15) | |
| Eicosanoic acid | C20:0 | 0.00 |
| Eicosanoic acid | C20:1 (cis-11) | 0.00 |
| Eicosadienoic | C20:2 | 0.00 |
| acid | (cis-11, 14) | |
| | C21:0 | 0.00 |
| Arachidonic | C20:4 | 0.00 |
| acid | | |
| Eicosapentenoic | C20:5 (cis-5, | 20.67 |
| acid (EPA) | 811, 14, 17) | |
| Docosanoic acid | C22:0 | 0.00 |
| Erucic acid | C22:1 (cis-13) | 0.00 |
| | C22:2 | 0.00 |
| | (cis-13, 16) | |
| Docosahexenoic | C22:6 (cis-4, 7, | 26.41 |
| acid (DHA) | 10, 13, 16, 19) | |
| Lignoceric acid | C24:0 | 0.00 |
| Nervonic acid | C24:1 (cis-15) | 0.00 |

Table 7 shows FA profiles of freshly tested tuna and chicken, SM-ES and SM-ES subjected to a tuna protocol of fat accumulation as described above. This is actually a repeat of the experiment shown in Table 6 ((A, DHA, EPA).

TABLE 7

| | | | | | SMES cells |
|---|---|---|---|---|---|
| | | | | | subjected to tuna |
| | | | | | differentiation |
| Fatty acid | | Chicken | Tuna | SMES | protocol |
| Palmitic acid | C16:0 | 20.68 | 19.1 | 22.4 | 17.28 |
| Palmitoleic acid | C16:1 (cis-9) | 3.42 | 3.76 | 5.53 | 3.97 |
| Stearic acid | C18:00 | 9.15 | 5.9 | 11.38 | 8.49 |
| Oleic acid | C18:1 (cis-9) | 32.47 | 12.6 | 33.13 | 24.09 |
| Linoleic acid | C18:2 (cis 9, 12) | 18.74 | 1.21 | 0.14 | 0 |
| eicosanoic acid | C20:1 (cis-11) | 0.41 | 0.96 | 1.94 | 0.9 |
| A rachidonic acid | C20:4 | 3.27 | 0 | 0.92 | 0.99 |
| eicosanoic acid (EPA) | C20:5 (cis-5, 8, | 0 | 6.52 | 0 | 5.14 |
| | 11, 14, 17) | | | | |
| Erucic acid | C22:1(cis-13) | 0 | 0 | 0.31 | 0 |
| Docosa hexenoic | C22:6(cis-4, 7, | 0.3 | 28.12 | 0.74 | 26.05 |
| acid (DHA) | 10, 13, 16, 19) | | | | |

Tuna values-The British journal of nutrition 103(2):189-96, DOI:10.1017/S0007114509991590

### Example 2

### Imparting Bovine Fatty Acid Profile to Avian Pluripotent Stem Cells

The Rational stands behind the following protocols (Example 2-4) is to prime the cells to adopt adipocyte identity and by this to open the possibility to adjust the fatty acid profile in a way that will create a fatty acid profile that is corresponding to the profile exist in the target species. An exemplary protocol is provided in Table 8*which can also be regarded as part of the specification and not just the examples section).

TABLE 8
Protocols for rendering the fatty acid profile of an avian
stem cell (e.g., SM-ES) to any of the following livestock
adipocytes (numbers are relevant to the absence of serum)

| FA | Name | LAMB | | BOVINE | | PORK | |
|---|---|---|---|---|---|---|---|
| Palmitic acid | C16:0 | 10-60 | uM | 10-60 | uM | 10-60 | uM |
| Stearic acid | C18:0 | 15-50 | uM | 5-15 | uM | 5-15 | uM |
| Oleic acid | C18:1 | 10-100 | uM | 10-100 | uM | 10-100 | uM |
| Linoleic acid | C18:2 | 0.1-5 | uM | 0.1-5 | uM | 5-20 | uM |

A stem cell line or in an embodiment of t e invention “the SM-ES” SuperMeat Culture meat cells) refers to a cell line of embryonic stem cells of avian origin, that can be in the form of isolated cells or aggregates as long as it contains stem cells. Embryonic stem cells can be driven to undergo cellular differentiation to all three lineages.

### Materials and Methods

Cells: cells used in the following examples originated from a proprietary line termed “SM” which are chicken established embryonic stem cells generated from a day 0 (un-incubated) fertilized egg. Cells were propagated in culture after removal from the egg to a plates in a DMEM F12 (Gibco #11320033)/10% FBS (Gibco #26140079) medium supplemented with 5 ng/L h-LIF (Peprotech #300-05). Followed by adaptation to serum free F12 based media.

Fatty acid analysis: analysis of fatty acids was done using HPLC-based methods. Analysis was performed using outsourced services (Laboratory for analytics and environmental development-Tel-Aviv university) Lipids staining: lipids staining was performed using BODIPY (Thermo-Fisher D3922) at a concentration of 1:1000.

### Generating a Bovine-Like Profile in Avian Stem Cells:

Pluripotent stem cells aggregates (SM) were seeded in a 500 ml flask at approximately 3*10⁶ cells/ml in priming Fat differentiation media (P-FDM) containing DMEM F-12 serum free media, Insulin 10 ug/ml (SARTORIUS, Cat. BE02-033E) Transferrin 10 ug/ml (PEPROTECH, Cat.10-366), Palmitic acid 20 uM (TCI, Cat. P1145), Stearic acid 15 uM (TCI, Cat. S0163). The flasks were incubated for 24 h at 39° C. and 5% C02, 95 rpm. Following 24 h (Day-1) Palmitic acid 60 uM (TCI, Cat. P1145), Stearic acid 50 uM (TCI, Cat. S0163), Linoleic acid 10 uM (ACROS, Cat. 60-33-3) and Oleic acid 25 uM (spectrum, Cat. 112-80-1) were added to the cells for a complete Fat differentiation media-Bovine (FDM-B). Flasks were incubated for 24 h at 39° C. and 5% C02, 95 rpm. After another 48h (Day-2), cells are observed under the microscope. Mature adipocytes should appear rounded with large lipid droplets apparent in the cytoplasm. Differentiated aggregates are stained for a final determination of fat cells using a lipid marker-Bodipy (1:1000) (Invitrogen, Cat. D3922), for 1 h at room temperature (RT).

### Results

The present inventors conducted a thorough analysis of fatty acid profile on the SM-ES cells. In this analysis, the treated cells (according to Table 7) were harvested and send to analysis for relative percentage of fatty acids. The profiles are shown in Table 9 below.

TABLE 9

| FA | Name | Chicken | Bovine | SM |
|---|---|---|---|---|
| Palmitic acid | C16:0 | 23.6% | 26.1% | 25.4% |
| Stearic acid | C18:0 | 6.9% | 14.5% | 17.2% |
| Oleic acid | C18:1 | 38.8% | 33.4% | 30.0% |
| Linoleic acid | C18:2 | 19.9% | 4.13% | 1.5% |

The table shows FA profile of tested SM-ES cells treated with Bovine fatty acid mix protocol compared to Chicken and Bovine published relative percentages.

### Example 3

### Imparting LAMB Fatty Acid Profile to Avian Pluripotent Stem Cells

### Generating a Bovine-Like Profile in Avian Stem Cells:

Pluripotent stem cells aggregates (SM) were seeded in a 500 ml flask at approximately 3*10⁶ cells/ml in priming Fat differentiation media (P-FDM) containing DMEM F-12 serum free media, Insulin 10 ug/ml (SARTORIUS, Cat. BE02-033E) Transferrin 10 ug/ml (PEPROTECH, Cat. 10-366), Palmitic acid 20 uM (TCI, Cat. P1145), Stearic acid 20 uM (TCI, Cat. S0163). The flasks were incubated for 24 h at 39° C. and 5% C02, 95 rpm. Following 24 h (Day-1) Palmitic acid 60 uM (TCI, Cat. P1145), Stearic acid 55 uM (TCI, Cat. S0163), Linoleic acid 10 uM (ACROS, Cat. 60-33-3) and Oleic acid 15 uM (spectrum, Cat. 112-80-1) were added to the cells for a complete Fat differentiation media-Lamb (FDM-L). Flasks were incubated for 24 h at 39° C. and 5% C02, 95 rpm. After another 48 h (Day-2), cells are observed under the microscope. Mature adipocytes should appear rounded with large lipid droplets apparent in the cytoplasm. Differentiated aggregates are stained for a final determination of fat cells using a lipid marker-Bodipy (1:1000) (Invitrogen, Cat. D3922), for 1 h at room temperature (RT).

### Example 4

### Imparting Pork Fatty Acid Profile to Avian Pluripotent Stem Cells

### Generating a Pork-Like Profile in Avian Stem Cells:

The Rational stands behind the following protocols is to prime the cells to adopt adipocyte identity and by this to open the possibility to adjust the fatty acid profile in a way that will create a fatty acid profile that is corresponding to the profile exist in the target species.

Pluripotent stem cells aggregates (SM) were seeded in a 500 ml flask at approximately 3*10⁶ cells/ml in priming Fat differentiation media (P-FDM) containing DMEM F-12 serum free media, Insulin 10 ug/ml (SARTORIUS, Cat. BE02-033E) Transferrin 10 ug/ml (PEPROTECH, Cat. 10-366), Linoleic acid 10 uM, Palmitic acid 20 uM (TCI, Cat. P1145) and Stearic acid 15 uM (TCI, Cat. S0163). The flasks were incubated for 24 h at 39° C. and 5% CO2, 95 rpm. Following 24 h (Day-1) Palmitic acid 60 uM (TCI, Cat. P1145), Stearic acid 50 uM (TCI, Cat. S0163), Linoleic acid 10 uM (ACROS, Cat. 60-33-3) and Oleic acid 25 uM (spectrum, Cat. 112-80-1) were added to the cells for a complete Fat differentiation media-Pork (FDM-P). Flasks were incubated for 24 h at 39° C. and 5% CO2, 95 rpm. After another 48 h (Day-2), cells are observed under the microscope. Mature adipocytes should appear rounded with large lipid droplets apparent in the cytoplasm. Differentiated aggregates are stained for a final determination of fat cells using a lipid marker-Bodipy (1:1000) (Invitrogen, Cat. D3922), for 1 h at room temperature (RT).

The patent or application file contains at least one drawing executed in color. Copies of this patent or patent application publication with color drawing(s) will be provided by the Office upon request and payment of the necessary fee.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of producing a food ingredient, the method comprising:
(a) providing stem cells; and
(b) culturing said stem cells in the presence of an effective amount of fatty acids or precursor thereof selected to reach an intracellular fatty acid profile of an edible species of interest which is not of the stem cells or adipocytes differentiated therefrom, so as to result in a food ingredient having a lipid organoleptic property of said edible species.

2. The method of claim 1, wherein said stem cells are embryonic stem cells, embryonic stem cells, adult stem cells or multipotent stem cells.

3. The method of claim 2, wherein said adult stem cells are of the mesoderm lineage.

4. The method of claim 2, wherein said adult stem cells are mesenchymal stem cells.

5. The method of claim 1, wherein said stem cells are selected from the group species of chicken stem cells and duck stem cells.

6. The method of claim 1, wherein said stem cells are in suspension.

7. The method of claim 1, wherein said stem cells are single cells.

8. The method of claim 1, wherein said stem cells are cell aggregates or microtissues.

9. The method of claim 1, wherein said culturing comprises a priming step followed by a differentiation step.

10. The method of claim 9, wherein a concentration of fatty acids in a medium of said priming step is low to ensure commitment to a mesodermal lineage or adipogenic fate but still maintains said stem cells in a proliferative stage.

11. The method of claim 10, wherein said concentration does not exceed 100 μM in the presence of serum or does not exceed 25 uM in the absence of serum.

12. The method of claim 9, wherein said priming step is 1-4 weeks long;
wherein a concentration of fatty acids in a medium of said differentiation step is above 100 μM in the presence of serum or is above 25 μM in the absence of serum; and/or
wherein said priming step is 1-14 days long.

13. The method of claim 1, wherein said intracellular fatty acid profile is as set forth in Tables 1 or 2.

14. The method of claim 1, wherein said precursor comprises acetyl-coA.

15. The method of claim 1, wherein said precursor is selected from the group consisting of citrate, malonate, isocitrate and palmitic acid.

16. The method of claim 1, wherein said culturing is effected in a serum-free medium.

17. The method of claim 9, wherein said priming and/or differentiation steps are performed in the presence of insulin and transferrin.

18. Cells obtainable according to the method of claim 1.

19. Cells comprising a genome of a first edible species and an intracellular fatty acid profile of a second edible species which is not of the first edible species.

20. A method of producing food, the method comprising combining the cells of claim 18 with an edible composition for human consumption.
